SEQUENCE LISTING (13 sequences)

SEQ ID NO: 1 - protein, 427 aa, Ilumatobacter sp.
MLTLMDLDAAVTSARASFVAAHPEAATWSDRARRVQPGGNTRSVLHVDPFPIRVDRAEGKHLWDLDGHRYVDLLGNYTAGLLGHSPEPVLAAARAALESGWSLGAVHENEVRLAELIVERFPSLDQVRFTNSGTEANMMALAVATHHTGRRKVVVFRNGYHGGVLTFGAEPSPVTVPHDWVLCDFNDLDSVSAAFAEHGVEIAAVLVEPMQGSGGCIPGTPAFLAGLRSLCDDHGALLVFDEVMTSRFSTGGAQQLLGVQPDMTTLGKYLAGGLTFGAFGGRADVMANFDPAGGTLAHAGTFNNNVASMAAGVAALTEVLSPELLDEVHARGERLRVRLNEAFAAAGLPMCATGVGSLMNVHGTAGPVGTAADLADQDDRLRELFYFHCLANGYYIARRGLIALSIEITDDDIDQFLDVVGSFGTDD

SEQ ID NO: 2 - DNA, 1296 bp, synthetic construct
  source 1..1296: mol_type = other DNA
catatgctgaccctgatggatctggacgcagcagttaccagcgcacgcgcaagttttgttgcagcacatccggaagcagcaacctggtctgatcgcgcacgtcgcgttcaaccgggcggtaatacccgttctgttctgcacgttgatccgtttccgattcgcgttgatcgcgcagaaggtaaacatctgtgggatctggacggtcatcgttacgttgatctgctgggtaactataccgcaggtctgctgggtcatagtccggaaccggttctggcagcagcacgcgcagcactggaatctggttggagtctgggcgcagttcacgaaaacgaagttcgtctggcagaactgatcgttgaacgttttccgagcctggatcaggtacgttttaccaacagcggtaccgaagccaatatgatggcactggcagttgcaacccatcataccggtcgtcgtaaagtcgtcgtctttcgcaacggctatcatggcggtgttctgacctttggcgcagaaccgagtccggttaccgttccgcacgattgggttctgtgcgatttcaacgacctggatagcgttagcgcagcatttgcagaacacggcgttgaaattgcggcagttctggttgaaccgatgcaaggttctggcggttgtattccgggtaccccggcatttctggcaggtctgcgttctctgtgcgatgatcacggcgcactgctggtatttgacgaagtcatgaccagccgtttttctaccggtggcgcacaacaactgctgggcgttcaaccggatatgaccaccctgggtaaatatctggcaggtggtctgacctttggcgcatttggcggtcgcgctgacgttatggcgaattttgatccggcagcaggtggtaccctggcacacgcaggcacctttaacaacaacgtcgcgtctatggcagcaggtgttgcagcactgaccgaagtactgagtccggaactgctggacgaagttcacgcacgcggcgaacgtctgcgcgtacgtctgaatgaagcatttgcagctgctggtctgccgatgtgtgcaaccggggttggttctctgatgaatgttcacggtaccgcaggtccggtaggtaccgcagcagatctggcagatcaagacgatcgtctgcgcgaactgttttacttccattgcctggcgaacggttattatattgcacgtcgcggtctgattgcgctgagcattgaaatcaccgacgacgatatcgatcagtttctggacgtggttggcagctttggtaccgacgattgactcgag

SEQ ID NO: 3 - DNA, 97 bp, synthetic construct
  source 1..97: mol_type = other DNA
aaacatctgtgggatctggacggtcatcgttacgttgatctgctgggtaactataccgcaggtctgctgggtcatagtccggaaccggttctggcag

SEQ ID NO: 4 - DNA, 97 bp, synthetic construct
  source 1..97: mol_type = other DNA
aaacatctgtgggatctggacggtcatcgttacgttgatccgctgggtaactatagcgcaggtctgctgggtcatagtccggaaccggttctggcag

SEQ ID NO: 5 - DNA, 97 bp, synthetic construct; identical to SEQ ID NO: 4
  source 1..97: mol_type = other DNA

SEQ ID NO: 6 - DNA, 97 bp, synthetic construct; identical to SEQ ID NO: 4
  source 1..97: mol_type = other DNA

SEQ ID NO: 7 - DNA, 101 bp, synthetic construct
  source 1..101: mol_type = other DNA
aaacatctgtgggatctggacggtcatcgttacgttgatccgctgggtaactatagcgcaggtctgctgggtcatagtccggaaccggttctggcagcagc

SEQ ID NO: 8 - DNA, 104 bp, synthetic construct
  source 1..104: mol_type = other DNA
aaacatctgtgggatctggacggtcatcgttacgttgatccgctgggtaactatagcgcaggtctgctgggtcatagtccggaaccggttctggcagcagcacg

SEQ ID NO: 9 - DNA, 97 bp, synthetic construct
  source 1..97: mol_type = other DNA
ctgccagaaccggttccggactatgacccagcagacctgcgctatagttacccagcggatcaacgtaacgatgaccgtccagatcccacagatgttt

SEQ ID NO: 10 - DNA, 97 bp, synthetic construct; identical to SEQ ID NO: 9
  source 1..97: mol_type = other DNA

SEQ ID NO: 11 - DNA, 97 bp, synthetic construct; identical to SEQ ID NO: 9
  source 1..97: mol_type = other DNA

SEQ ID NO: 12 - DNA, 101 bp, synthetic construct
  source 1..101: mol_type = other DNA
gctgctgccagaaccggttccggactatgacccagcagacctgcgctatagttacccagcggatcaacgtaacgatgaccgtccagatcccacagatgttt

SEQ ID NO: 13 - DNA, 104 bp, synthetic construct
  source 1..104: mol_type = other DNA
cgtgctgctgccagaaccggttccggactatgacccagcagacctgcgctatagttacccagcggatcaacgtaacgatgaccgtccagatcccacagatgttt

(19) United States
(12) Patent Application Publication
(10) Pub. No.: US 2025/0043292 A1
(43) Pub. Date: Feb. 6, 2025

(54) INSILICO GUIDED CRISPR-CAS DRIVEN ENZYME ENGINEERING FRAMEWORK

(71) Applicant: Kcat Enzymatic Private Limited, Bangalore (IN)

(72) Inventors: Pravin Kumar R, Bangalore (IN); Dipa NATARAJAN, Bangalore (IN); Gladstone Sigamani G, Bangalore (IN); Roopa L, Bangalore (IN)

(73) Assignee: Kcat Enzymatic Private Limited, Bangalore, KA (IN)

(21) Appl. No.: 18/804,157

(22) Filed: Aug. 14, 2024

Related U.S. Application Data
(63) Continuation of application No. PCT/IB2022/051451, filed on Feb. 18, 2022.

(51) Int. Cl. C12N 15/70; C12N 9/22; C12N 15/11; G16B 20/20

(52) U.S. Cl. CPC C12N 15/70; C12N 9/22; C12N 15/11; G16B 20/20; C12N 2310/20; C12N 2800/101

(57) ABSTRACT

The invention describes a method for utilizing the CRISPR-Cas system to edit any gene of interest present on a plasmid. The method uses CRISPR tool to engineer enzymes for better activity by allowing a cell to undergo specific and random mutations. Described methods include newly designed, engineered and modified vector systems, which encodes single or multiplex gene targets and Cas9/deaminase Cas9 proteins. The invention is useful for single or multiple gene editing for industrial applications such as to edit genes encoding antibiotics, therapeutic proteins or any important industrial enzymes. The invention is a quick and efficient tool for creating enzyme variant libraries containing a vast range of permutation and combination of mutation that will be assayed for highest activity. Hot spots will be identified on gene of interest which will aid in generating mutations in these places. These mutations can be rationalized in specific places or random single base substitutions.

### FIELD OF INVENTION

In nature enzymes have the ability to mutate but at a low rate based on their environment. However, iIn industry, enzymes are mutated (engineered) for improved efficiency and high activity, increased productivity and to work in different conditions such as extreme or varying temperatures, pH and solvents. A major hiccup in engineering enzymes is, that the residues chosen for mutations are limited due to difficulties involved in screening. The present invention is a method of enzyme engineering for creating enzyme libraries. The method provides a quick and efficient tool for creating libraries containing a vast range of permutation and combination of mutation that will be assayed for highest activity.

### BACKGROUND OF THE INVENTION

CRISPR Cas technology is used extensively for genome editing with which genomes are manipulated by Knock-ins and knock-outs, gene therapy corrections as well as for single nucleotide substitutions. As a technology, it has been much studied and improved for various studies. Almost all the editing described are at the genome level of either prokaryotes or eukaryotes, plants and microbes. The technology can be used to mutate several genes at the same time as well as target several regions of a gene at the same time. Therefore, we aim to use CRISPR Cas technology to introduce different permutations of appropriate mutations to generate a library of variants.

### SUMMARY OF THE INVENTION

The invention describes the methods of directing CRISPR complex formation in bacterial cells and a novel method for utilizing the CRISPR-Cas system to edit gene of interest present on a plasmid. Described methods include newly designed and modified vector systems, which encodes single or multiplex gene targets and Cas9/deaminase Cas9 proteins. The invention discloses a methodology to introduce combination of specific and random mutation in a gene of interest in the plasmid DNA of a bacterial cell. Initially, hotspots for mutations are identified within the gene, encompassing areas for specific mutations and designated regions for random mutagenesis in the enzyme that the gene encodes. These mutations are programmed into sgRNAs and donor DNA, which are then incorporated into engineered pCas9 plasmids. A key feature of this invention is the construction of a first engineered pCas9 plasmid, which includes a J23119 promoter preceding the sgRNAs, a lac promoter before the gene for the engineered dCas9 enzymes, and a temperature-inducible lambda operator before both elements. The dCas9 gene in this plasmid is modified from its natural form and combined with a gene for an engineered deaminase enzyme to enhance activity. The process continues with the transformation of a bacterial cell with this plasmid, introducing the gene of interest and the engineered pCas9 system into the cell. Subsequently, a second engineered pCas9 plasmid is introduced to the cell, aimed at inducing specific mutations identified earlier. This second plasmid carries an engineered Cas9 enzyme gene, additional sgRNAs and donor DNAs matching the specific mutations, regulated by a leader sequence and a tet promoter. Mutation to the Cas9 enzyme gene include deletions and substitutions to reduce the enzyme's size and enhance its activity. The claimed invention utilizes a synergistic combination of engineered dCas9 enzymes with deaminase activity and engineered Cas9 enzymes contained in separate plasmids to induce specific and random mutations within a gene at designated regions. This approach allows the gene to remain intact in the plasmid inside bacterial cell, facilitating mutation without the need for gene extraction, simplifying the process to one or two steps. This not only represents a substantial improvement over traditional methods but also provides a more efficient, precise and expedited means for creating mutation libraries, marking a transformative advance in genetic engineering for enzyme engineering. The invention is useful for single or multiple gene editing for industrial applications for example to edit genes encoding antibiotics, therapeutic proteins or any important industrial enzymes. The invention is a quick and efficient tool with few number of steps for creating enzyme variant libraries containing a vast range of permutation and combination of mutations that will be assayed for highest activity.

### BRIEF DESCRIPTION OF DRAWINGS

FLOWCHART 1: The flow chart describes overall experiments that will be followed to give a permutation/combination of specific and random mutations to achieve variants with best functionality. Curled arrows depict steps in the process ranging from steps 1 to 7.

FIG. 1: Prediction of hotspots using insilico studies: The figure shows a schematic representation of the hotspots predicted for an enzyme, example, transaminase using insilico studies. However, the representation is not just limited to transaminase, the insilico methodology, can be applied for any protein of interest, enzymes, therapeutic proteins, antibodies, extended drug discovery process.

FIG. 2: Schematic diagram of CRISPR guided Cas9 system: An overview of the experiment. Represents the design to incorporate mutations into gene of interest which is present on a plasmid.

FIG. 3: Inhouse-engineered Crispr/Cas9 and inhouse-engineered Crispr/dCas9 enzymes with deaminase activity for incorporating random and specific mutation simultaneously in Bacteria

FIG. 4: Strategy for introducing mutations into gene of interest with iterations. FIG. 4A Shows first round of experiments where transformation is done and competent cells are produced. FIG. 4B Shows the next set of transformations using in silico selected regions for incremental mutations

FIG. 5: Schematic diagram of sequential gene editing with dCas9 and pCas9 and two-plasmid system.

FIG. 6: Incorporating 2 or 3 PLASMID systems in a cell for one-step mutagenesis of heterologous gene: Cloning engineered Cas9 and engineered dCas9 (eCas9 and edCas9) in single vector or co-transforming simultaneously. FIG. 6A Shows eCas9 and edCas9 in single vector. FIG. 6B Shows eCas9 and edCas9 in separate vector co-transformed into cell carrying pET28

FIG. 7: Schematic strategy for cloning multiple guide RNAs into individual plasmids as well as together into a single plasmid.

FIG. 8: Schematic strategy for cloning site specific guide RNAs and their donor templates into single plasmid

FIG. 9: A first engineered pCas9 plasmid construction and mechanism targeting random mutations at multiple target sites. FIG. 9A Shows the engineered pCas9 plasmid expresses dCas9 fused with an engineered deaminase, sgRNA, and RNA scaffold. FIG. 9B Shows the combination of all CRISPR elements forms a CRISPR/dCas9 deaminase complex.

FIG. 9C Shows the dCas9 deaminase mediates multiple random base mutations at multiple targeted sites within 100 base pairs of PAM sites of the gene of interest present on the pET-28a(+) plasmid inside bacterial cells.

FIG. 10: Illustrates the mechanism of second engineered pCas9 plasmid: FIG. 10A Shows the engineered pCas9 plasmid expresses an engineered Cas9 enzyme, sgRNA, trans activating RNA and donor DNAs. FIG. 10B Shows the combination of all CRISPR elements forms a CRISPR/Cas9 complex. FIG. 10C Shows the Cas9 mediates multiple specific mutations at multiple targeted sites within 3 base pairs of PAM sites of the gene of interest present on the pET-28a(+) plasmid inside bacterial cells.

FIG. 11: Expected permutation/combination of mutations mediated by CRISPR/Cas9 generating a library of variants

FIG. 12: Preliminary results of testing the transformation of 2 plasmids into bacteria.

FIG. 13: Colorimetric based enzyme assay to check activity of variants.

### DEFINITIONS

### Customized Vector

The current approach outlines the application of engineered CRISPR/Cas9 or engineered CRISPR/dCas9 systems within a bacterial system.

The term “a customized vector” is replaced with “an engineered plasmid”. The engineered plasmid consists of an engineered Cas9 along with one or more guide RNAs and one or more donor templates, and another containing an engineered dCas9 fused with deaminase alongside one or more guide RNAs.

To facilitate the expression of the components mentioned above, we have developed distinct engineered plasmids tailored for Cas9-mediated specific mutations and dCas9-mediated random mutations, as detailed in section 0081 of the specification. Below, we elaborate on these customized vectors.

Engineered dcas9 Enzymes:

The gene for engineered dCas9 enzymes is designed to carry mutations relative to the wild-type dCas9, enhancing its binding capabilities without cleavage activity. This engineered dCas9 is then fused with a gene for a deaminase enzyme, which also contains mutations compared to its natural counterpart, to boost its activity. This fusion results in a powerful tool for precise gene editing, allowing for targeted deamination and thus base editing, without cutting the DNA, offering a refined approach to gene modification.

“One or More sgRNAs.”
3.1 A First Engineered pCas9 Plasmid with One or More sgRNAs:

‘One or more sgRNAs’ are one or more guide RNA sequences which are cloned together in a single plasmid in order to incorporate one or more random mutations into a gene of interest located within the plasmid DNA. These guide RNAs are short synthetic RNA sequences that guide the engineered dCas9 enzymes to the specific target sequences within the target gene where it induces random mutations without double strand break. They are designed to be complementary to the DNA sequence adjacent to the target site, directing the engineered dCas9 enzyme to bind and to induce the mutation at that precise location.

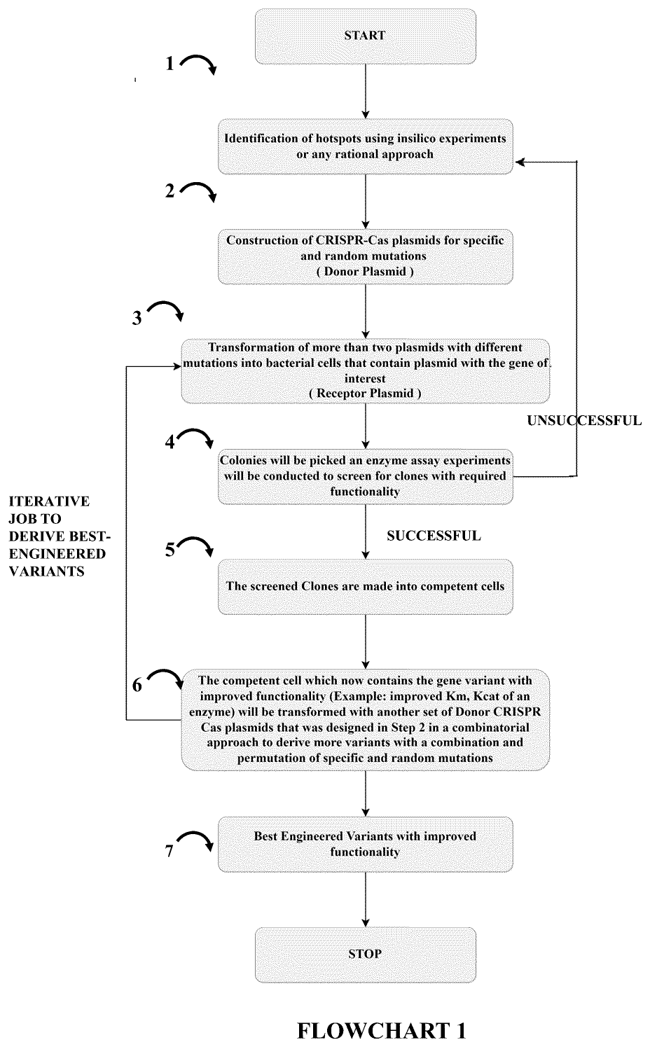

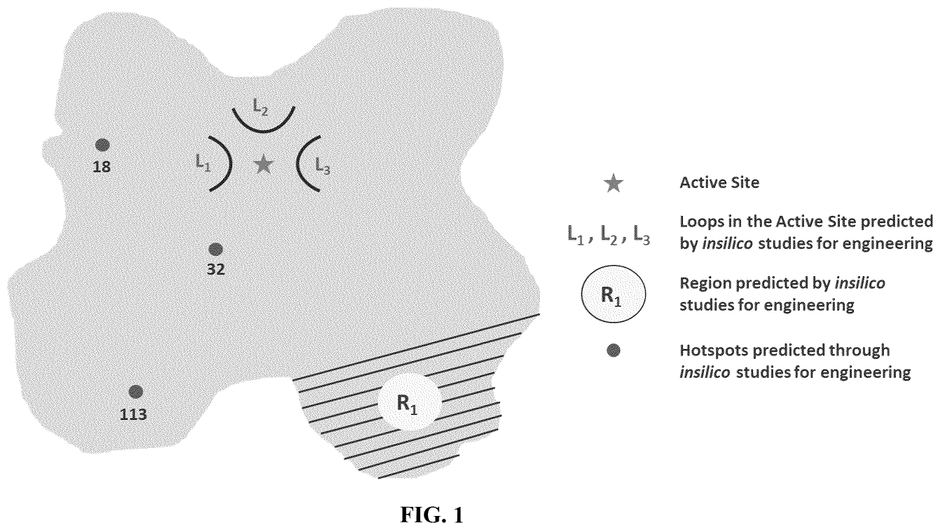

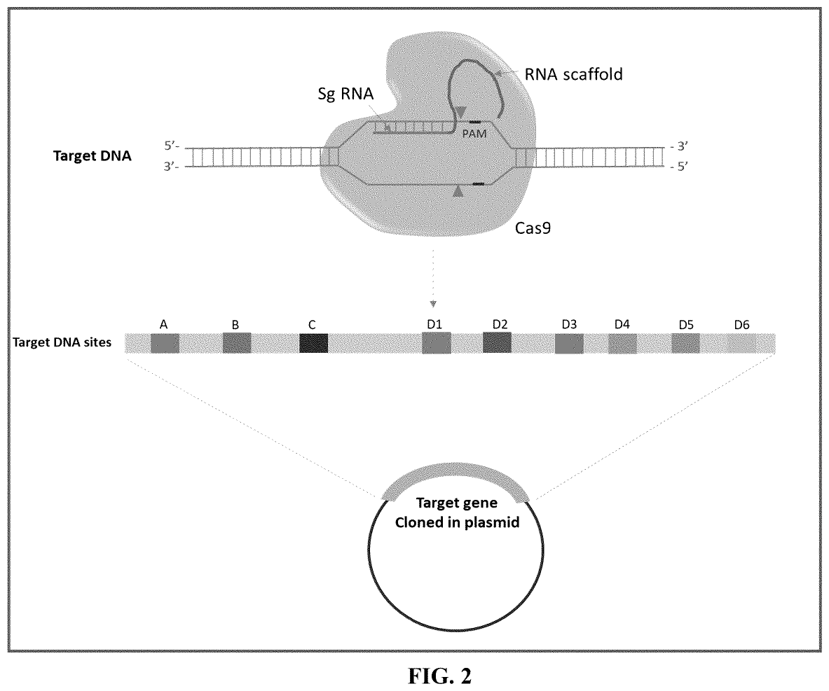

In this context, “one or more sgRNAs” refers to the targeting of multiple sites for mutation in certain experiments. To accomplish this, one or more sgRNAs have been cloned into a single plasmid. For instance, a first engineered pCas9 plasmid is designed in such a way where one or more sgRNAs can be cloned between two direct repeats and expressed under the regulation of J23119 promoter. In order to induce engineered dCas9 enzyme mediated random mutation near target site 1, designed guide RNA is labeled as sgRNA D1 (FIG. 1)) for target site 2, designed guide is named as sgRNA D2 (FIG. 2); for target site 3, it's designated as sgRNA D3 (FIG. 3); and for target site 4, it's labeled as sgRNA D4. All of these guide RNA from D1 to D4 are carrying the complementary gene sequences to the corresponding regions where random mutations are desired. Upon the transformation, all of these sgRNAs (D1 to D4) gets expressed, each of this sgRNA will form separate binding complex containing engineered dcas9 enzyme fused with deaminase enzyme and will guide it to the corresponding region for desired random mutation.

3.2. Components of the First Engineered pCas9 Plasmid for Random Mutations:

Origin of replication: A DNA sequence of pSC101 origin to initiate the replication of plasmid components in the bacterial cell

Rep101 protein coding sequence: It is temperature sensitive Rep101 protein, required to initiate replication with pSC101 origin

lac UV5 promoter: This sequence initiates and regulates the expression of dCas9 enzymes.

a. In the First Engineered pcas9 Plasmid, the Expression of Dcas9 Enzyme is Driven by Lac UV5 Promoter

Engineered dcas9 enzyme encoding sequence: The engineered dcas9 enzyme encoding sequence is mutated to increase its stability and efficiency inside the bacterial system

J23119 Promoter sequence: In the given engineered plasmid, expression of one or more sgRNAs and RNA scaffold sequence are driven by J23119 promoter.

One or more sgRNAs—multiple guide RNAs can be cloned between the direct repeat sequences to target one or more target sites.

In this context, “one or more sgRNAs” refers to the targeting of multiple sites for mutation in certain experiments. To accomplish this, one, two, three, or more sgRNAs have been cloned into a single plasmid. For instance, to induce dCas9-deaminase mediated random base mutation near target site 1, the guide RNA is labeled as sgRNA D1; for target site 2, it's named sgRNA D2; for target site 3, it's designated as sgRNA D3; and for target site 4, it's labeled as sgRNA D4.

Scaffold RNA sequence: It pairs with each of the repeat RNA sequences within the precursor transcript, forming double-stranded RNA (dsRNA). RNase III then cleaves at the dsRNA repeat generated by the scaffold RNA to produce the efficient CRISPR-dCas9 complex.

Promoter for antibiotic resistance: In the described plasmid ampicillin gene expression is driven by ampicillin resistance gene specific promoter

Antibiotic resistance encoding gene sequence: beta-lactamase encoding gene sequence which confers the ampicillin resistance

3.3. Construction and Mechanism of Targeting Random Mutations at Multiple Target Sites of the First Engineered pCas9 Plasmid

The first Engineered pdCas9 plasmid is constructed by replacing existing cas9 enzyme encoding sequence with an in house engineered deactivated cas9 (dcas9) enzyme encoding gene sequence in order to achieve random mutations. This plasmid is constructed using pScI_dCas9-CDA_J23119 (available in addgene) as base plasmid. All the necessary regulatory components gene sequences such as J23119 promoter, temperature inducible lamda repressor, lac UV5 promoter and pSC101 origin were derived from pScI_dCas9-CDA_J23119. Engineered dcas9 enzyme coding sequence is synthesized and cloned under the regulation of lacUV5 promoter and temperature inducible lambda repressor, where the expression of dcas9 can be controlled by changing the temperature during cell growth in order to regulate the intensity of random mutations. At downstream of dcas9 gene an engineered cytidine deaminase or adenine deaminase sequences are added during the synthesis of dcas9 gene sequence so that dcas9 enzyme will be fused with cytidine or adenine deaminase enzyme in order to achieve base editing for random mutations in vivo.

The existing plasmid pScI_dCas9-CDA_J23119 cannot facilitate the cloning of more than one guide RNAs. In the present engineered pCas9 plasmid, we have added direct repeat sequences with bsaI sites to clone one or more sgRNAs in a single cloning step. Where expression of one or more sgRNAs is carried out by J23119 promoter. At further downstream of the guideRNA array, modified RNA scaffold sequence is cloned in an engineered plasmid, where scaffold sequence will be expressed along with sgRNAs and bind to form an efficient crispr dcas9 complex.

The existing plasmid pScI_dCas9-CDA_J23119 contains chloramphenicol resistance gene sequence driven by cat promoter. In an engineered pdCas9 plasmid this sequence is replaced with ampicillin resistance gene sequence derived from commercially available pUC19 plasmid driven by ampicillin resistance specific promoter sequence.

Upon transformation into bacterial cells, an engineered pCas9 plasmid expresses all necessary CRISPR elements in a temperature-controlled manner. When the cell culture temperature surpasses 39° C., the lambda repressor enzyme becomes inactive, triggering the expression of dCas9 enzymes fused with cytidine or adenine deaminase. Simultaneously, multiple sgRNAs are expressed, binding with RNA scaffold and dCas9-deaminase enzyme to form a CRISPR/dCas9 deaminase complex, as depicted in FIG. 1B. This multi CRISPR/dCas9 deaminase complex, guided by multiple sgRNAs, targets numerous sites on the target gene located on the pET-28a(+) plasmid within the bacterial cell. Unlike Cas9, deactivated Cas9 (dCas9) does not induce mutations via double-strand breaks; instead, it facilitates point mutations through fused cytidine/adenine deaminase, converting C to T or A to G within 100 base pairs of PAM sites. The positive clones will be screened on antibiotic selection medium.

3.4. A Second Engineered pCas9 Plasmid with One or More sgRNAs:

One or more sgRNAs' are one or more guide RNA sequences which are cloned together in a single plasmid in order to incorporate one or more specific mutations into a gene of interest located within the plasmid DNA. These guide RNAs are short synthetic RNA sequences that guide the Cas9 enzyme to desired specific target sequences within the target gene where it induces double strand break and do the desired mutations encoded in donor DNA. They are designed to be complementary to the DNA sequence adjacent to the target site, directing the Cas9 enzyme to bind to and cleave the DNA at that precise location. In this context, when we refer to “one or more sgRNAs,” it signifies that in certain experiments, multiple target sites have been selected for mutation. To accomplish this, a second engineered pCas9 plasmid contains one or more sgRNAs cloned along with one or more donor DNA under the regulation of leader sequence. For instance, to induce a specific mutation at target site A, the guide RNA is named sgRNA A; for target site B, it's named sgRNA B; and for target site C, it's named sgRNA C. Each of these guide RNAs, containing a complementary sequence adjacent to multiple targeting sites, will form separate CRISPR-Cas9 complexes. These complexes will induce specific desired mutations at each of the targeted sites.

### One or More Donor DNAs

Here, we have utilized “one or more donor DNAs” to construct a second engineered pCas9 plasmid, as defined below:

4.1. A Second Engineered pCas9 Plasmid with One or More Donor DNAs:

“one or more donor DNAs” are one or more DNA templates cloned along with one or more sgRNAs into a second engineered pCas9 plasmid. Donor DNA templates carry a desired gene sequence intended to be precisely incorporated at a specific site within a target gene, located within the plasmid DNA. These donor DNAs serve as templates to incorporate the desired DNA sequence for mutation via homologous recombination.

In this context, when we refer to “one or more donor DNAs,” it signifies targeting multiple specific homology-directed repair mutations in certain experiments. To accomplish this, one or more donor DNAs have been cloned into a second engineered pCas9 plasmid with one or more donor DNAs. For instance, to induce mutation A, the donor template is labeled as donor A; for mutation B, it's named donor B; and for mutation C, it's designated as donor C. All of these donor DNAs, containing two flanking homology arms adjacent to the target DNA region, will serve as templates for incorporating multiple desired mutations at multiple sites.

4.2. Components of an Engineered pCas9 Plasmid for Specific Mutations:

Origin of replication: A particular DNA sequence p15 A origin to initiate the replication of plasmid components in the bacterial cell

Promoter sequence: This sequence initiates and regulates the expression of Cas9 protein enzyme. In second engineered pcas9 plasmid, the expression of cas9 enzyme is driven by tet promoter

Engineered cas9 enzyme encoding sequence: It is truncated and mutated cas9 enzyme coding sequence to increase its stability and efficiency inside the bacterial system

Leader sequence: It initiates the transcription of one or more sgRNAs

One or more sgRNAs: multiple guide RNAs can be cloned between the direct repeat sequences to target one or more target sites.

In this context, when we refer to “one or more sgRNAs,” it signifies that in certain experiments, multiple target sites have been selected for mutation. To accomplish this, one, two, three, or more sgRNAs have been cloned into a single plasmid. For instance, to induce a specific mutation at target site A, the guide RNA is named sgRNA A; for target site B, it's named sgRNA B; and for target site C, it's named sgRNA C.

One or more donor templates: multiple donor DNAs sequences are designed to achieve desired mutations through homologous recombination at CRISPR cas9 target site.

In this context, when we refer to “one or more donor DNAs,” it signifies targeting multiple specific homology-directed repair mutations in certain experiments. To accomplish this, one, two, three, or more donor DNAs have been cloned into a single plasmid. For instance, to induce mutation A, the donor template is labeled as donor A; for mutation B, it's named donor B; and for mutation C, it's designated as donor C.

Tracr RNA sequence, It is a necessary CRISPR element needed for RNA scaffolding to form CRISPR cas9 binding complex

Promoter for antibiotic resistance: In the described plasmid chloramphenicol gene expression is driven by cat promoter

Antibiotic resistance encoding gene sequence: Chloramphenicol acetyltransferase encoding gene sequence which confers the Chloramphenicol resistance

4.3. Second Engineered pCas9 Plasmid Construction and Mechanism for Targeting Specific Mutations:

The second Engineered pCas9 plasmid is constructed by replacing existing cas9 enzyme encoding sequence with an in house engineered cas9 enzyme encoding gene sequence in order to make the crsipr/cas9 system more efficient. All the necessary CRISPR elements, tracr RNA and leader sequence were amplified from pCas9 plasmid (available in addgene) and cloned together with synthesized engineered cas9 gene sequence. Synthesized tet promoter sequence is cloned at upstream of engineered cas9 gene sequence to regulate the proteinenzyme expression. Leader sequence is cloned at upstream of the CRISPR array comprising one or more sgRNAs and one or more donor DNAs. To facilitate the multiplex cloning of all these oligos of guide RNAs and donor DNAs, bsaI site is introduced between two direct repeats. One or more sgRNAs and one or more donor DNAs are cloned between direct repeats by using golden gate assembly (as shown in FIG. 2). Synthesized tet promoter sequence is cloned upstream of engineered cas9 gene sequence to regulate the enzyme expression. Chloramphenicol resistance gene and promoter sequence is derived from pCas9 and cloned in an engineered pCas9 plasmid as a selection marker.

Upon transformation into the bacterial cell, all CRISPR elements are expressed, leading to the formation of a CRISPR/Cas9 complex. This complex binds to the respective target sites on the target gene present on the expression plasmid. Each CRISPR/Cas9 complex targets a specific site, guided by sgRNA. Cas9 induces double-stranded cleavage within 3 base pairs upstream of the PAM region. The damaged DNA strand is repaired through homology-directed recombination. The donor template contains flanking arms at both the 5′ and 3′ ends, providing the desired mutation template to insert desired DNA sequence at the target location.

### DETAILED DESCRIPTION

### Manipulating Plasmid DNA in Prokaryotes Using CRISPR for Enzyme Engineering

Mutations will be incorporated onto gene of interest (example Transaminases), already cloned in plasmids and transformed into bacterial cells where the manipulation includes a mix of site specific mutations as well as random mutations (flow chart 1).

### Mutations Designed by Insilico Studies

Combination of rational site-specific mutations and random mutations. Using an engineered Cas vector, single base mutations designed from in silico studies will be employed to to introduce mutations onto gene present on a plasmid using CRISPR-Cas technology.

The regions for manipulations by CRISPR-Cas either specific or single base substitutions performed with deaminase, dCas9 (DAM and labelled here as D) is selected from in silico studies or by just random selection.

Permutation/combination of mutations The combination is fed into a computer algorithm that implements AI such as genetic algorithm to increase the probability of permutations and combinations

Design of Mutations Introduced Sequentially onto Gene of Interest for Generating Library of Engineered Enzymes (Example Transaminase).

The following steps will be used:
a. Incorporating specific mutations and random mutations using CRISPR-Cas technology either in separate plasmids or in one plasmid thereby using either a 2 step process or a 1 step process respectively.
b. Evaluate enzyme activity by using screening assay protocols for selecting colonies with good activity.
c. The variants showing good activity will be used as the starting point for the next iteration

### Analyses of Colonies in Enzyme Library:

These will be done either by adding substrate to the media or on agar plates or in 96 well plates and assessing activity using a colorimetry assay; or by extracting the enzyme from lysate and performing colorimetry/HPLC.

Colonies with good activity will be sequenced to confirm combination and location of mutations.

Computational Studies to Fine Tune the Combinations—Optimisation Using the Training Dataset Derived from CRISPR-Cas

1. Making competent cells from transformed cells in order to increase number of variants which will increase the number of mutations in the gene in increments.

In order to get a higher percentage of targeted or random hits, a small number of plasmids (3-4) with a mixture of specific and random mutations (single base substitutions) will be transformed.

The analyzed clones will be made competent and subjected to second and third rounds of transformation with plasmids with different mutations.

In this way, we can ensure that each round will add mutations to the gene of interest without overwhelming the system.

The colonies/clones will be assayed for activity of protein or enzyme and the ones with highest activity will be sequenced to assess mutations.

Different concentration of plasmids transformed to give different outcomes and combinations.

If all plasmids of same size are transformed at same concentration, the probability of all added mutations in various plasmids being transferred to the gene of interest would be equal.

If plasmids are transformed at different concentrations then probability of plasmids with higher concentration transferring mutation may be higher than that of lesser concentration based on formula:
X=transformation threshold
Dam1, 2&3, 4>X
Plasmid intake Factor=a

We will assess how the region of homology of target site—GC or AT rich will affect this probability

Experiments will decipher optimum concentration which could be that the mutations on plasmids at lower concentration would work better.

### Multiple Target Sites in a Single Transformation to Give Different Mutations in a Single Gene

Constructs will be designed with up to 6-8 mutations including rationalized specific mutations and random base substitutions.

These experiments will be compared for activity of protein or enzyme and resulting gene sequenced for assessment.

The mutations will be designed to be controlled and target specific regions of the gene.

The region of interest will be targeted with specific and random mutations in order to create focussed library of permutation combination of mutations

### Use of CRISPR-Cas9 Method to Generate Specific and Random Mutation in Single Experiment

Plasmid construct and design will be based on regions of gene chosen by insilico studies to introduce mutations. The region of the gene with the mutations will be synthesized and cloned along with their sgRNA and donor template into engineered pCas9 or dCas plasmids which will then be transformed into bacterial cell containing the gene of interest on a plasmid.

### Addition of Single Base Editor for Random Substitutions as Opposed to Random Mutagenesis Experiments

The current alternative to genome editing is base editing, the conversion of one base pair into another without requiring the creation and repair of double strand breaks.

Using insilico studies, we describe a method whereby engineered Deaminase random base editing technology with CRISPR Cas9 system will be used to introduce base changes in specific regions. This engineered dCas9 along with Cas9 will result in mutations in specific regions in 2 or 3 steps.

### Multi Plasmid CRISPR/Cas System

This is a marker free gene editing method to introduce specific and random mutations by transforming 3-4 plasmids.

Each plasmid will be at different concentration to give multiple permutation/combination. In this way, we can ensure ed multiple sites of mutations in a single step.

All variants that show good or high activity as an outcome from all the different versions of the experiments will be sequenced and will be fed back into the AI to derive the next round of experiments.

Based on these, new set of sgRNAs, Donor DNAs, pCas9/dCas9 plasmids will be designed and experiments performed to derive more focused libraries till the highest enzyme activity and conversion rates are reached.

### Description of Methodology in Detail

### Step 1

Prediction of hotspots (FIG. 1) using insilico studies of the enzyme transaminase as an example. However, the representation is not just limited to transaminase, the insilico methodology, can be applied for any protein of interest, enzymes, therapeutic proteins, antibodies, extended drug discovery process etc.

In FIG. 1, the positions 18, 32, 113 are hotspot derived from insilico studies and the substitutions predicted by insilico studies are A18C, D32S, T113Y. This region is labelled as core substitutions
‘A’ ‘B’ ‘C’
A18C [Core 1], D32S [Core 2], T113Y [Core 3]

From the MD, QM/MM or other insilico studies a region is chosen, which is considered a promising region, which upon engineering would yield better activity. The region is from 180 to 205 region (R₁) position; loops that are converging in the active site 6-12 (L₁), 38-45 (L₂), 118-152 (L₃)

The Core 1 [A], Core 2 [B], Core [C] will be incorporated by CRISPR Cas in combination with L₁, L₂, L₃ and Region 1 [R₁]
Where,
L₁→D1
L₂→D2
L₃→D3
R₁→D4
Core 1 [A], Core 2 [B], Core [C] mutations will have “Defined Substitutions”;
L₁, L₂, L₃ & Region 1 [R₁] will be used as region to incorporate “Random Substitutions”

The steps for insilico studies are given below:

MD simulations or QM/MM simulations, Ensemble Docking, NMA of the protein of the Enzyme-Substrate, Enzyme—Intermediate state and Enzyme product simulation is executed.

Use MD trajectory to predict substrate diffusion and product efflux/egress using tools such as CAVER, trj cavity etc.

Use Kcat Contact Score algorithm to predict Hotspots by capturing the residue that came in contact with the substrate.

Use SSM, PHP and 7D Grid Technology to predict substitutions.

The PHP Engine is a probe-based screening process that is used to generate substitutions, permutations and combinations of substitutions over hotspot residues that is present in the active site.

The above insilico studies will be conducted to predict hotspots and substitutions.

As shown and described in FIG. 1 (example—transaminase), using these predictions, in the wet lab, CRISPR-Cas experiments will be used to introduce mutations both specific through rational design as well as random via activated deaminase mediated single base editing.

These experiments, divided broadly into 4 steps (explained in detail in FIGS. 3-10) will not only incorporate mutations but also create a permutation/combination of mutations to make versatile enzyme libraries.

The overall idea is to construct engineered pCas9 and dCas9 plasmids with a combination of specific mutations as well as random single base mutations, which when incorporated into the gene of interest will resemble the schematic image shown in FIG. 2 (as mentioned in Step (2) of Flow chart 1) wherein, there are specific mutations at designated places and random ones within certain regions.

### Step 2

FIG. 2 represents the design to incorporate mutations into gene of interest which is present on a plasmid. The Cas9 nuclease (in pink) will target specific sites on the gene cloned in a plasmid (light blue) by an sgRNA consisting of approximately 20-nucleotide guide sequence (red) and a scaffold (brown). The guide sequence pairs with the specific target sites on the gene (blue bar between the strands), directly 3-4 nucleotide upstream of a 5′-NGG adjacent motif (Protospacer Adjacent Motif, PAM; navy blue). Cas9 will generate a double stranded break ˜3 bp upstream of the PAM (shown as a red triangle) that activates repair mechanism to disrupt or mutate DNA sequences at or near the cleavage site. Here we represent two types of mutations that will be incorporated into the gene of interest. All mutations are depicted as coloured squares on a target gene sequence represented by light blue line. From insilico studies, specific sites will be chosen for specific mutations here represented as A, B and C. Sites for random mutations will also be designed using insilico studies and will be introduced using activated deaminase for single base editing with an engineered defective Cas (dCas9) which will allow incorporation of mutations without double strand breaks. Although these are shown here as all mutations on the gene, there will be permutation and combinations of these mutations as explained in detail in FIGS. 3 to 10.

### Step 3

Introducing Mutations into Gene of Interest with Iterations:

FIG. 3 describes a process for introducing multiple mutations into a gene of interest located within Plasmid DNA that is transformed into a bacterial cell using engineered CRISPR-Cas technology, the process comprising:
identifying hotspots in the gene of interest where the hotspots include specific mutations and designated regions for random mutations in the enzyme encoded by the gene of interest wherein, the respective mutations are encoded in donor DNA and guided by the sgRNA that are inserted in engineered pCas9 plasmids;

2. characterized in that the process comprises:
constructing a first engineered pCas9 plasmid with one or more sgRNAs that corresponds to the regions for random mutagenesis derived from step (a), and the engineered pCas9 plasmid contains a J23119 promoter upstream of the sgRNAs and a lac promoter upstream of the gene encoding engineered dCas9 enzymes, and a temperature-inducible lambda operator upstream of both components, wherein the gene encoding the engineered dCas9 enzymes are mutated with reference to the naturally occurring dCas9 and is fused with a gene encoding deaminase enzyme containing mutations with reference to the naturally occurring gene encoding deaminase enzyme;
transforming a bacterial cell with the first engineered pCas9 plasmid obtained in step b resulting in the bacterial cell being transfected with both, the plasmid DNA carrying the gene of interest and the first engineered pCas9 plasmid; and
transforming a second engineered pCas9 plasmid into the bacterial cell obtained in step c, to induce multiple specific mutations in the gene of interest in the plasmid DNA, wherein the second engineered pCas9 plasmid contains a gene encoding engineered Cas9 enzyme, one or more sgRNAs, and one or more donor DNAs that corresponds to the specific mutations obtained in step (a), and is characterized by a leader sequence upstream to the sgRNAs and donor DNAs and a tet promoter upstream of the gene encoding engineered Cas9 enzyme, wherein the gene encoding engineered Cas9 enzyme has deletions and substitution mutations with reference to the naturally occurring gene that encodes Cas9 enzyme.

Alternatively mutations can be induced also by the following steps
identifying hotspots in the gene of interest in the Plasmid DNA of the bacterial cell; characterized in that the process comprises:
constructing one or more specific mutations using pCas9 vector or customized vector, wherein each of the specific mutations are different and expressed in different pCas9 vectors or customized vectors, wherein the customized vector comprises engineered Cas9 enzymes and one or more sgRNA's in combination with one or more donor DNA's;
constructing one or more random mutations using customized vector or dCas9 vector, wherein each of the random mutations are different and expressed in different dCas9 vectors or customized vectors, wherein the customized vector comprises an engineered dCas9 enzyme and one or more sgRNA's;
transforming the vectors comprising the specific mutations and the vectors comprising random mutations together in the bacterial cell;
transforming competent bacterial cell with the vector comprising specific mutation and the vector comprising random mutation, wherein the specific mutations and random mutations are different from the specific and random mutations mentioned in step d;
repeating the steps c and d one or more times to incorporate more numbers of specific and random mutations expressed in the bacterial cell; and
obtaining the bacterial cell comprising combination of multiple specific mutations and random mutations.

Individual plasmids carrying either specific mutations for example for sites A, B, C, or random mutations with the combination of Deaminase (depicted as D1 to D6) are constructed along with their sgRNAs, spacers and either Cas9 or dCas9 or our customized engineered vectors based on insilico studies.

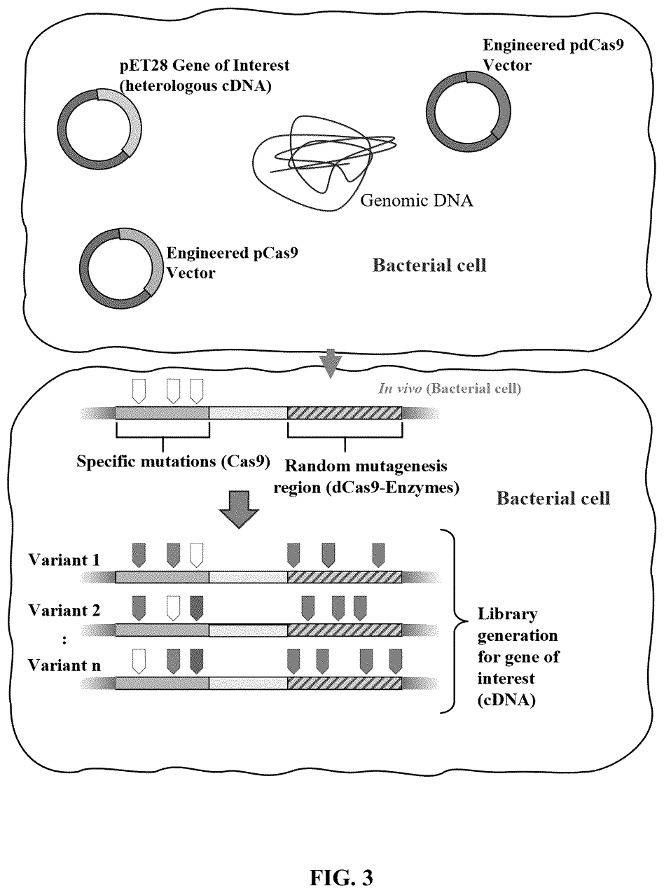

This is shown on the top row as circles with specific colours representing different mutations (these are the Donor plasmids). (Figure. 4)

There are 4 main steps—1 to 4 that explain the procedure of Step 3.

### Part A

Step a: Firstly, a selection of 3 (or more) plasmids carrying either specific mutations (A, B or C) or random mutations constructed with deaminase (D1-D6) as shown with arrows under circles will be transformed into competent parental bacterial line containing the gene of interest on a plasmid.

This is depicted here as a tube with light grey filling (this is the receiving plasmid).

Step b: After transformation using conventional methods, the cells will be plated onto agar plates with appropriate antibiotic selection.

Step c: From these plates 4 to 6 or more colonies will be picked and cultured. These will be used for assays to verify the enzyme activity.

This will be performed using different methods including colorimetry as shown by different coloured tubes.

Step d: Colonies showing good or high levels of enzyme activity will be prepared for next Round.

They will be made into competent cells and these will be used for methods shown in part B.

### Part B:

### Next Round of Transformations Using in Silico Selected Regions for Incremental Mutations:

The process in part B will use competent cells made from colonies with gene of interest now with mutations introduced in previous transformation.

As shown, another selection of plasmids with specific mutations (A, B or C) or random mutations (depicted as D1-D6 in circles) in part B will be transformed into newly made competent cells.

These will be processed through Steps 1-4 again for Round 2.

### Step 4

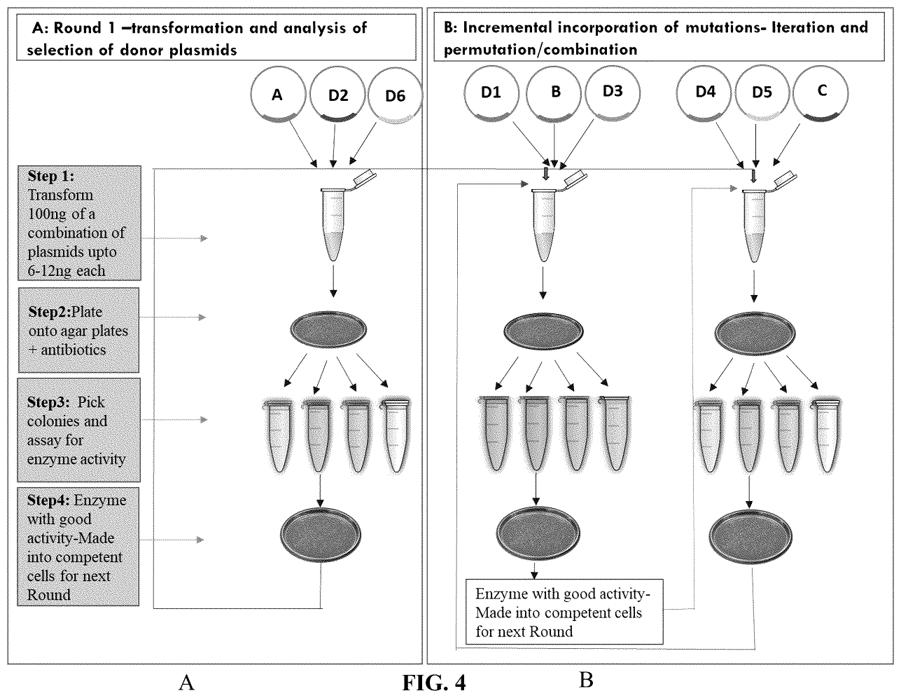

FIG. 5 shows cchematic diagram of sequential gene editing with dCas9 and Cas9 and two-plasmid system.

All random single base mutations by deaminase method will be cloned into one plasmid along with their respective sgRNAs and engineered dCas9 or our customized vector and all site-specific mutations will be cloned into one plasmid along with their respective sgRNAs, donor templates and Cas9.

Step 1 of FIG. 5 shows the generated plasmid construct with all the sgRNAs targeted for deaminase dCas9 mediated mutations (donor plasmid).

Step 2 of FIG. 5 shows transformation of this plasmid in bacterial cell with gene of interest on a plasmid (Receiving plasmid).

These will be plated onto agar plates with antibiotics specific for both (or more) plasmids.

Colonies will be screened and assayed for enzyme activity as well as for mutations incorporated.

Also, colonies can be maintained on kanamycin selection medium to deactivate dCas9.

In step 3 of FIG. 5, selected screened clones will be made into competent cells and transformed with pCas9 plasmid or our engineered customized vectors carrying sgRNAs for site-specific mutations with their respective donor templates.

Positive clones will be screened for enzyme activity and sequenced to evaluate the permutation and combination of mutations.

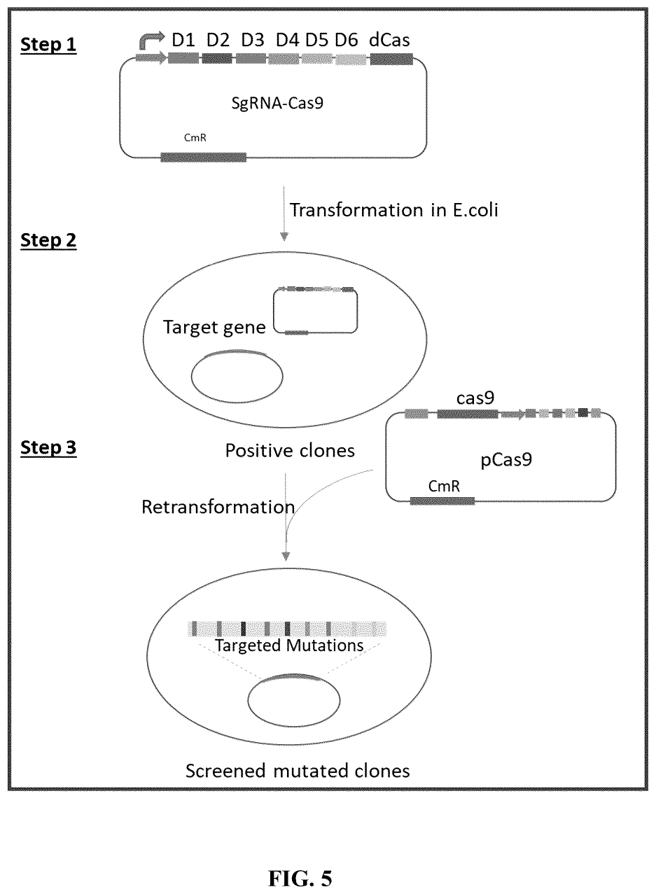

FIG. 6 shows Incorporating 2 or 3 PLASMID systems in a cell for one-step mutagenesis of heterologous gene. This is achieved by Cloning engineered Cas9 and engineered dCas9 (eCas9 and edCas9) in single vector or co-transforming simultaneously. The vector design or transformation of plasmids into the bacterial system is targeted to work on a heterologous gene that is co-transformed with this system. This way the process generates enzyme variants in a single step or maximum two steps.

### Step 5

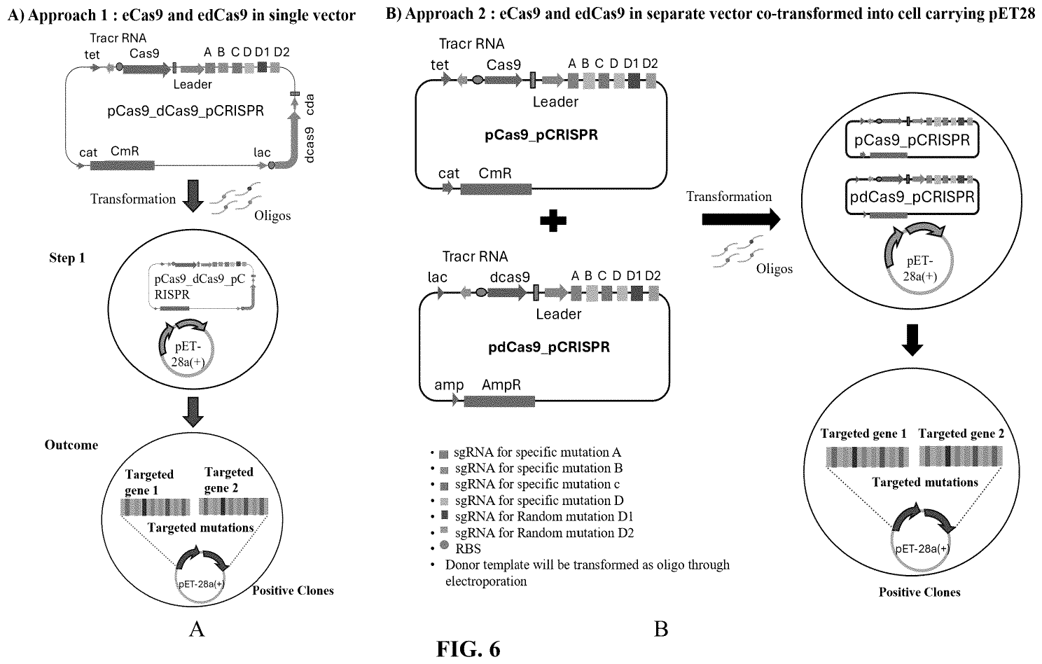

FIG. 7. gives details of cloning strategy.

A vector, for example, pScI_dCas9-CDA_J23119 or our customized plasmid which expresses sgRNA scaffold (small green rectangle on plasmid) driven by J23119 promoter upstream of the sgRNAs and a lac promoter upstream of the gene encoding engineered dCas9 enzymes and cytosine deaminase fused dCas9 or our customized plasmid (engineered dCas9 enzymes mutated with reference to the naturally occurring dCas9 and is fused with a gene encoding deaminase enzyme containing mutations with reference to the naturally occurring gene encoding deaminase enzyme) under a temperature-inducible lambda operator upstream of both components, can be used.

The individual large rectangles represent plasmids and individual sgRNA for target sites are depicted in different coloured small rectangles as sgRNA1, sgRNA2 etc to sgRNA6.

Step 1 of FIG. 7 shows six target sgRNAs oligonucleotides designed and synthesized and annealed together.

These are digested with appropriate restriction enzymes to clone into digested pScI_dCas9-CDA_J23119 with same restriction enzymes.

Step 2 of FIG. 7 represents plasmids with sgRNAs and scaffold cassette together ligated into vector with BsaI restriction enzyme for directional cloning of multiplex sgRNAs.

These plasmids are individual plasmids carrying individual mutations for various sites.

In Step 3 of FIG. 7, the same vector, for example, pScI_dCas9-CDA_J23119 plasmid or our customized engineered vector will be used as a multiplex sgRNA expression vector.

All six sgRNAs cassettes for various sites will be digested with BsaI and ligated to generate multiplex D1 to D6 target RNAs and dCas9 using Golden Gate technology.

These are donor plasmids as described in Step (2) of Flow chart 1.

### Step 6

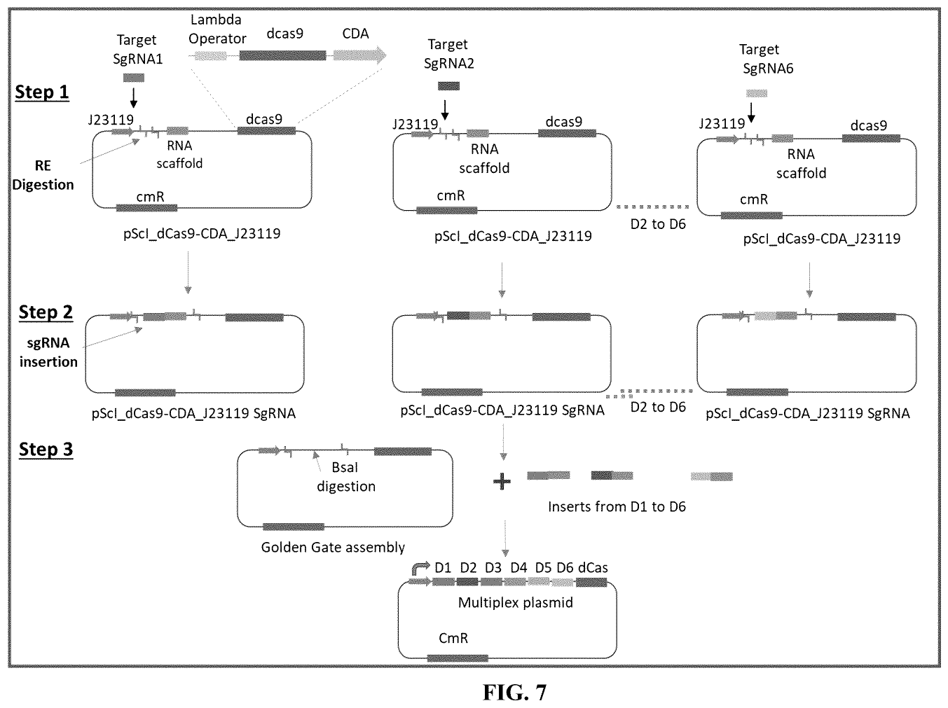

FIG. 8 shows schematic strategy for cloning site-specific guide RNAs and their templates into single plasmid; The figure represents the cloning strategy for target site-specific sgRNAs. The sgRNAs will be designed using insilico studies, synthesized along with their templates as shown here as different coloured strips, A, B and C. These specific oligonucleotides will be digested using specific restriction enzyme, BsaI, and sgRNAs will be cloned into a pCas9 or our customized engineered vector along with the templates also digested with BsaI.

In order to clone all sgRNAs together, golden gate assembly will be used and all the digested sgRNAs will be cloned into pCas9 or into our customized engineered vectors.

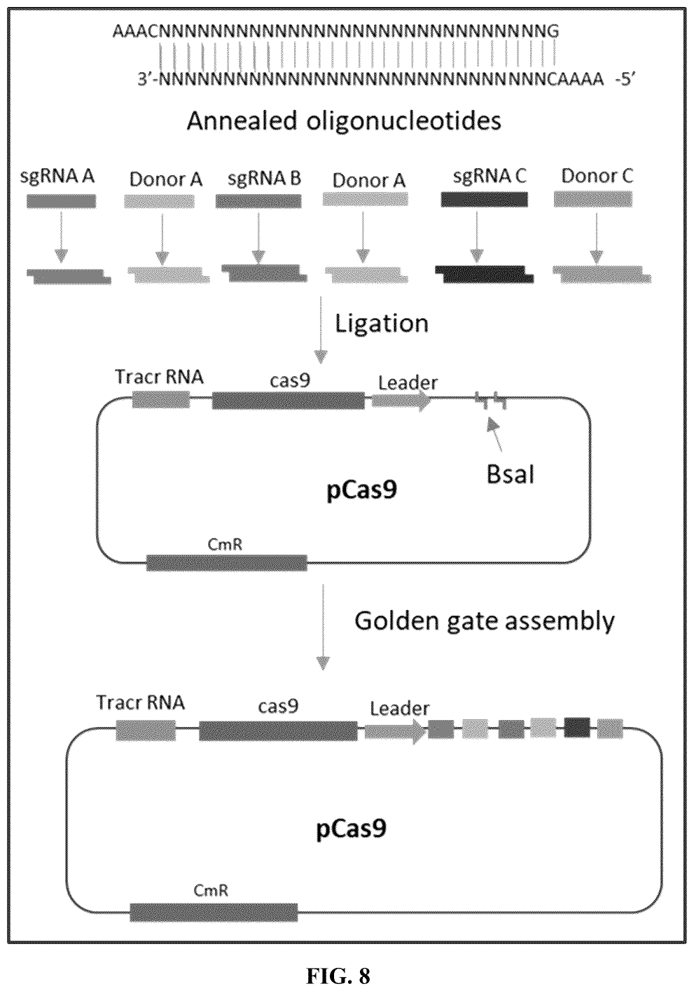

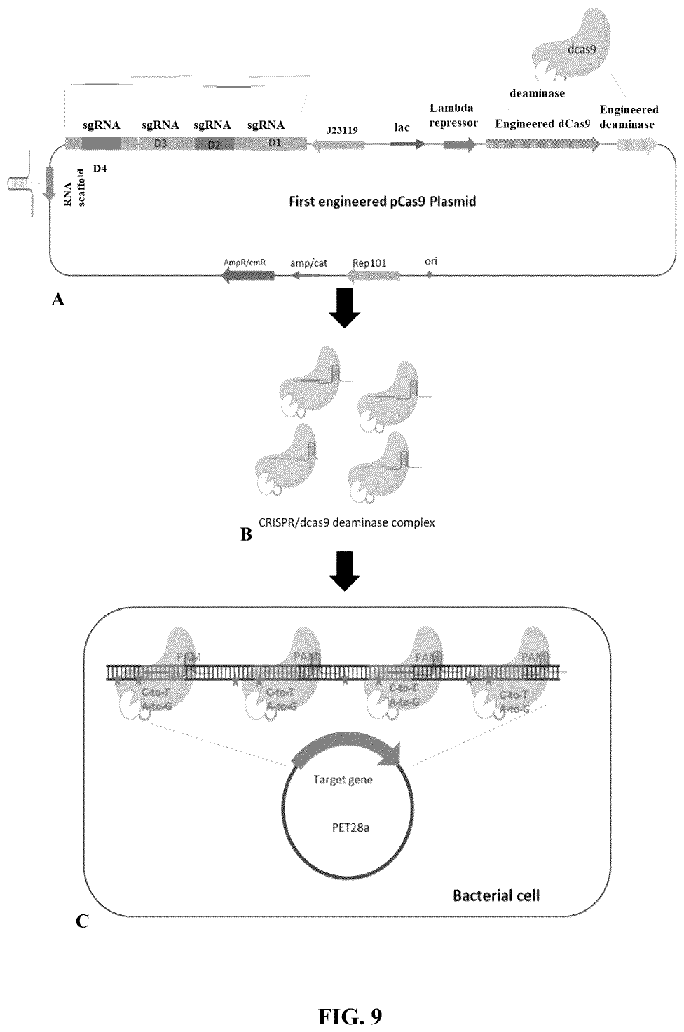

FIG. 9 explains construction and mechanism of targeting random mutations at multiple target sites of the first engineered pCas9 plasmid

The first Engineered pdCas9 plasmid is constructed by replacing existing cas9 enzyme encoding sequence with an in house engineered deactivated cas9 (dcas9) enzyme encoding gene sequence in order to achieve random mutations. This plasmid is constructed using pScI_dCas9-CDA_J23119 (available in addgene) as base plasmid. All the necessary regulatory components gene sequences such as J23119 promoter, temperature inducible lamda repressor, lac UV5 promoter and pSC101 origin were derived from pScI_dCas9-CDA_J23119. Engineered dcas9 enzyme coding sequence is synthesized and cloned under the regulation of lacUV5 promoter and temperature inducible lambda repressor, where the expression of dcas9 can be controlled by changing the temperature during cell growth in order to regulate the intensity of random mutations. At downstream of dcas9 gene an engineered cytidine deaminase or adenine deaminase sequences are added during the synthesis of dcas9 gene sequence so that dcas9 enzyme will be fused with cytidine or adenine deaminase enzyme in order to achieve base editing for random mutations in vivo.

The existing plasmid pScI_dCas9-CDA_J23119 cannot facilitate the cloning of more than one guide RNAs. In the present engineered pCas9 plasmid, we have added direct repeat sequences with bsaI sites to clone one or more sgRNAs in a single cloning step. Where expression of one or more sgRNAs is carried out by J23119 promoter. At further downstream of the guideRNA array, modified RNA scaffold sequence is cloned in an engineered plasmid, where scaffold sequence will be expressed along with sgRNAs and bind to form an efficient crispr dcas9 complex.

The existing plasmid pScI_dCas9-CDA_J23119 contains chloramphenicol resistance gene sequence driven by cat promoter. In an engineered pdCas9 plasmid this sequence is replaced with ampicillin resistance gene sequence derived from commercially available pUC19 plasmid driven by ampicillin resistance specific promoter sequence.

Upon transformation into bacterial cells, an engineered pCas9 plasmid expresses all necessary CRISPR elements in a temperature-controlled manner. When the cell culture temperature surpasses 39° C., the lambda repressor enzyme becomes inactive, triggering the expression of dCas9 enzymes fused with cytidine or adenine deaminase. Simultaneously, multiple sgRNAs are expressed, binding with RNA scaffold and dCas9-deaminase enzyme to form a CRISPR/dCas9 deaminase complex, as depicted in FIG. 1B. This multi CRISPR/dCas9 deaminase complex, guided by multiple sgRNAs, targets numerous sites on the target gene located on the pET-28a(+) plasmid within the bacterial cell. Unlike Cas9, deactivated Cas9 (dCas9) does not induce mutations via double-strand breaks; instead, it facilitates point mutations through fused cytidine/adenine deaminase, converting C to T or A to G within 100 base pairs of PAM sites. The positive clones will be screened on antibiotic selection medium.

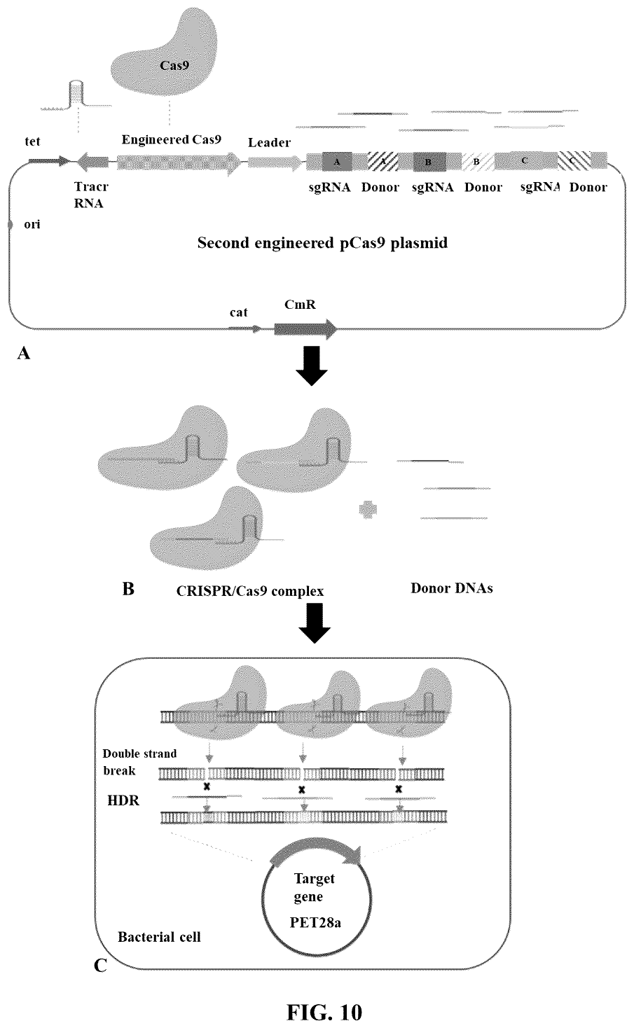

FIG. 10 explains the second engineered pCas9 plasmid construction and mechanism for targeting specific mutations. The second Engineered pCas9 plasmid is constructed by replacing existing cas9 enzyme encoding sequence with an in house engineered cas9 enzyme encoding gene sequence in order to make the crsipr/cas9 system more efficient. All the necessary CRISPR elements, tracr RNA and leader sequence were amplified from pCas9 plasmid (available in addgene) and cloned together with synthesized engineered cas9 gene sequence. Synthesized tet promoter sequence is cloned at upstream of engineered cas9 gene sequence to regulate the proteinenzyme expression. Leader sequence is cloned at upstream of the CRISPR array comprising one or more sgRNAs and one or more donor DNAs. To facilitate the multiplex cloning of all these oligos of guide RNAs and donor DNAs, bsaI site is introduced between two direct repeats. One or more sgRNAs and one or more donor DNAs are cloned between direct repeats by using golden gate assembly (as shown in FIG. 2). Synthesized tet promoter sequence is cloned upstream of engineered cas9 gene sequence to regulate the enzyme expression. Chloramphenicol resistance gene and promoter sequence is derived from pCas9 and cloned in an engineered pCas9 plasmid as a selection marker.

Upon transformation into the bacterial cell, all CRISPR elements are expressed, leading to the formation of a CRISPR/Cas9 complex. This complex binds to the respective target sites on the target gene present on the expression plasmid. Each CRISPR/Cas9 complex targets a specific site, guided by sgRNA. Cas9 induces double-stranded cleavage within 3 base pairs upstream of the PAM region. The damaged DNA strand is repaired through homology-directed recombination. The donor template contains flanking arms at both the 5′ and 3′ ends, providing the desired mutation template to insert desired DNA sequence at the target location.

### Step 7

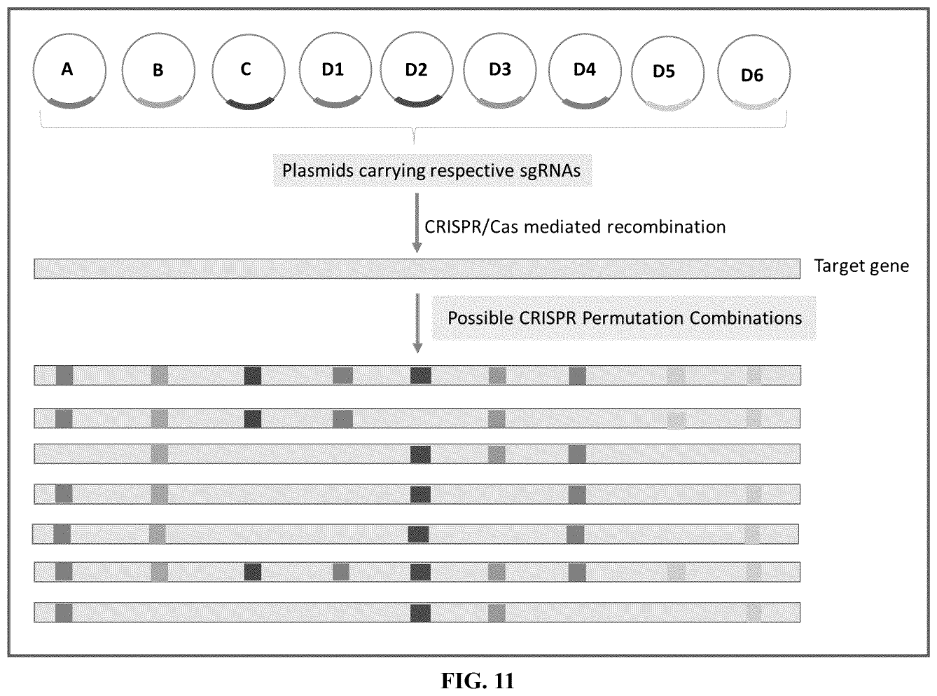

### FIG. 11: Expected Permutation Combinations Mediated by CRISPR/Cas9

The above process will result in mutations added on to the gene of interest in increments.

The possible outcomes are depicted in the FIG. 11.

Plasmids with site-specific mutations and random mutations with single base editing using deaminases or other methods are shown in circles (depicting donor plasmid DNA) with different coloured strips (depicting the different sgRNA regions along with mutations).

Upon transformation and analysis, the different CRISPR permutations that can be expected are shown in the light blue lines with coloured square regions matching the coloured strips on plasmid circles.

These represent the different permutation and combination of mutations—either specific or random, that can be expected after each round of transformation with different selection of plasmids as described in FIG. 4.

The permutation/combination of mutations is manifold.

This representation is an example of some of the possible outcomes.

The final round of generating permutation/combination of mutation will be defined by deriving an enzyme clone with highest activity (as Step (7) in Flow chart 1).

The specific mutations will be cloned into vectors carrying Cas9 protein too, whereas the random single base editing by activated deaminases (1) will be cloned into vectors carrying a defective Cas9 (dCas9) or customized vectors for Cas9 and dCas9 which allows mutations without double strand breaks.

Experiments will also involve constructing individual plasmids carrying either individual specific or random mutations and also constructing singe plasmids that carry all mutations either specific or random.

Here we use the example of enzyme Transaminase.

FIG. 4 describes the design and plan of experiment in detail for the incorporation of mutations into gene of interest (enzyme engineering of Transaminase).

This plan is based on individual plasmids constructed with individual mutations (these will be the donors).

These will be transformed into competent parental bacterial line containing the gene of interest on a plasmid (the receiving plasmid), mentioned in Flow chart 1 as steps (3) and (4).

Colonies will be picked up and the enzyme activity assessed using colorimetry tests or HPLC.

The colonies that show good activity will be used for the next round of transformations after being made competent again (mentioned as step (5) in Flow chart 1), with a different set of plasmids carrying random mutations on the sgRNA.

Successful colonies will be taken to next round (as in step (6) of Flow chart 1).

The process will be continued till incrementally all the designed mutations are incorporated and also highest enzyme activity is achieved (shown as step (7) in Flow chart 1).

### EXPERIMENTAL STUDIES AND RESULTS

Insilico design for finding mutation hotspots and designing sgRNAs and donor templates

>KCAT_4: WILD
MLTLMDLDAAVTSARASFVAAHPEAATWSDRARRVQPGGNTRSVLHVDPFPIRVDR
AEGKHLWDLDGHRYVDLLGNYTAGLLGHSPEPVLAAARAALESGWSLGAVHENE
VRLAELIVERFPSLDQVRFTNSGTEANMMALAVATHHTGRRKVVVFRNGYHGGVLT
FGAEPSPVTVPHDWVLCDFNDLDSVSAAFAEHGVEIAAVLVEPMQGSGGCIPGTPAF
LAGLRSLCDDHGALLVEDEVMTSRESTGGAQQLLGVQPDMTTLGKYLAGGLTFGAF
GGRADVMANFDPAGGTLAHAGTENNNVASMAAGVAALTEVLSPELLDEVHARGER
LRVRLNEAFAAAGLPMCATGVGSLMNVHGTAGPVGTAADLADQDDRLRELFYFHC
LANGYYIARRGLIALSIEITDDDIDQFLDVVGSFGTDD
>KCAT_4: Optimized (for E.coli) Sequence
CATATGCTGACCCTGATGGATCTGGACGCAGCAGTTACCAGCGCACGCGCAAGTT
TTGTTGCAGCACATCCGGAAGCAGCAACCTGGTCTGATCGCGCACGTCGCGTTCA
ACCGGGCGGTAATACCCGTTCTGTTCTGCACGTTGATCCGTTTCCGATTCGCGTTG
ATCGCGCAGAAGGTAAACATCTGTGGGATCTGGACGGTCATCGTTACGTTGATCT
GCtggGTAACTATACCGCAGGTCTGCTGGGTCATAGTCCGGAACCGGTTCTGGCAG
CAGCACGCGCAGCACTGGAATCTGGTTGGAGTCTGGGCGCAGTTCACGAAAACG
AAGTTCGTCTGGCAGAACTGATCGTTGAACGTTTTCCGAGCCTGGATCAGGTACG
TTTTACCAACAGCGGTACCGAAGCCAATATGATGGCACTGGCAGTTGCAACCCAT
CATACCGGTCGTCGTAAAGTCGTCGTCTTTCGCAACGGCTATCATGGCGGTGTTC
TGACCTTTGGCGCAGAACCGAGTCCGGTTACCGTTCCGCACGATTGGGTTCTGTG
CGATTTCAACGACCTGGATAGCGTTAGCGCAGCATTTGCAGAACACGGCGTTGA
AATTGCGGCAGTTCTGGTTGAACCGATGCAAGGTTCTGGCGGTTGTATTCCGGGT
ACCCCGGCATTTCTGGCAGGTCTGCGTTCTCTGTGCGATGATCACGGCGCACTGC
TGGTATTTGACGAAGTCATGACCAGCCGTTTTTCTACCGGTGGCGCACAACAACT
GCTGGGCGTTCAACCGGATATGACCACCCTGGGTAAATATCTGGCAGGTGGTCTG
ACCTTTGGCGCATTTGGCGGTCGCGCTGACGTTATGGCGAATTTTGATCCGGCAG
CAGGTGGTACCCTGGCACACGCAGGCACCTTTAACAACAACGTCGCGTCTATGGC
AGCAGGTGTTGCAGCACTGACCGAAGTACTGAGTCCGGAACTGCTGGACGAAGT
TCACGCACGCGGCGAACGTCTGCGCGTACGTCTGAATGAAGCATTTGCAGCTGCT
GGTCTGCCGATGTGTGCAACCGGGGTTGGTTCTCTGATGAATGTTCACGGTACCG
CAGGTCCGGTAGGTACCGCAGCAGATCTGGCAGATCAAGACGATCGTCTGCGCG
AACTGTTTTACTTCCATTGCCTGGCGAACGGTTATTATATTGCACGTCGCGGTCTG
ATTGCGCTGAGCATTGAAATCACCGACGACGATATCGATCAGTTTCTGGACGTGG
TTGGCAGCTTTGGTACCGACGATTGACTCGAG
##Designed Guide RNA: LEU73PRO_THR78SER
>Wild
AAACATCTGTGGGATCTGGACGGTCATCGTTACGTTGATCTGCTGGGTAACTATA
CCGCAGGTCTGCTGGGTCATAGTCCGGAACCGGTTCTGGCAG
###### Positive Strand #####
>D1+

| | | | | | Cut to |
|---|---|---|---|---|---|
| S. | | | | | mutation |
| No | Position | Strand | Sequence | PAM | distance |
| 01 | 204 | + | TCATCGTTACGTTGATCTGC | TGG | 0 |
| 02 | 205 | + | CATCGTTACGTTGATCTGCT | GGG | 0 |
| 03 | 220 | + | CTGCTGGGTAACTATACCGC | AGG | 0 |
| 04 | 239 | − | ACTATGACCCAGCAGACCTG | CGG | 4 |
| 05 | 228 | + | TAACTATACCGCAGGTCTGC | TGG | 7 |

AAACATCTGTGGGATCTGGACGGTCATCGTTACGTTGATCCGCTGGGTAACTATA
GCGCAGGTCTGCTGGGTCATAGTCCGGAACCGGTTCTGGCAG
>D2+
AAACATCTGTGGGATCTGGACGGTCATCGTTACGTTGATCCGCTGGGTAACTATA
GCGCAGGTCTGCTGGGTCATAGTCCGGAACCGGTTCTGGCAG
>D3+
AAACATCTGTGGGATCTGGACGGTCATCGTTACGTTGATCCGCTGGGTAACTATA
GCGCAGGTCTGCTGGGTCATAGTCCGGAACCGGTTCTGGCAG
>D4+
AAACATCTGTGGGATCTGGACGGTCATCGTTACGTTGATCCGCTGGGTAACTATA
GCGCAGGTCTGCTGGGTCATAGTCCGGAACCGGTTCTGGCAGCAGC
>D5+
AAACATCTGTGGGATCTGGACGGTCATCGTTACGTTGATCCGCTGGGTAACTATA
GCGCAGGTCTGCTGGGTCATAGTCCGGAACCGGTTCTGGCAGCAGCACG
#####Negative Strand ####
>D1−
CTGCCAGAACCGGTTCCGGACTATGACCCAGCAGACCTGCGCTATAGTTACCCAG
CGGATCAACGTAACGATGACCGTCCAGATCCCACAGATGTTT
>D2−
CTGCCAGAACCGGTTCCGGACTATGACCCAGCAGACCTGCGCTATAGTTACCCAG
CGGATCAACGTAACGATGACCGTCCAGATCCCACAGATGTTT
>D3−
CTGCCAGAACCGGTTCCGGACTATGACCCAGCAGACCTGCGCTATAGTTACCCAG
CGGATCAACGTAACGATGACCGTCCAGATCCCACAGATGTTT
>D4−
GCTGCTGCCAGAACCGGTTCCGGACTATGACCCAGCAGACCTGCGCTATAGTTAC
CCAGCGGATCAACGTAACGATGACCGTCCAGATCCCACAGATGTTT
>D5−
CGTGCTGCTGCCAGAACCGGTTCCGGACTATGACCCAGCAGACCTGCGCTATAGT
TACCCAGCGGATCAACGTAACGATGACCGTCCAGATCCCACAGATGTTT
Bold letters in sequences indicate Mutation Site (LEU73PRO_THR78SER, CTG>CCG, ACC>AGC)
Lower case letters indicate PAM site
Underlined letters indicate Donor DNA Region

### Methodology for Plasmid Design and Construction

Step 1. Selection of sgRNA Targets

sgRNA from different regions of gene will be designed using CRISPR guide tools.

The region of Protospacer Adjacent Motif (PAM) will also be selected and designed for all the regions either for specific mutations or for random mutations.

### Step 2: Synthesis of the Guide Oligonucleotides

Specific Forward and Reverse primers for each region will be designed including specific restriction enzymes.

Appropriate vectors with either dCas9 for random mutations or Cas9 for specific mutations will be selected as well as engineered customized pCas9 and dCas9 vectors that will be designed and synthesized.

A few will be tested for best results.

Step 3: Ligation into Vector

Each pair of oligo fragments will be phosphorylated and annealed together.

The appropriate vector will be digested with the same Restriction enzymes.

Then the two will be ligated together to get final construct.

The reaction mix of inserts (such as sgRNAs, templates and PAMs etc) and the vectors will be assembled using Golden Gate technology.

The end CRISPR-Cas plasmids constructs are depicted as circles with different colors in FIG. 4 also called Donor plasmids.

In FIG. 5, we describe the strategy to clone all the mutations either specific

or random in one relevant plasmid and how this will be used for further

experiments.

### Bacterial Strain and Plasmids

Appropriate plasmid vectors will be chosen and also the parent bacterial strain containing gene of interest will be selected.

This will be the receiving plasmid as shown as in FIG. 4 in grey tube.

The strain will be BL21 as a cloning host, for fast performance and to enable protein induction.

These will be analysed with restriction enzyme digests and run on agarose gel electrophoresis

Protocol for Sequential Transformation of Plasmids with Mutations and the Screening Procedure

Step 1: Transformation at the same time, of 10-100 ng of 2 or more plasmids carrying sequences to target either specific sites or random sites on the gene of interest or 1 plasmid with all mutations.

Gene of interest is in the parental line. Transformation will be done by heat shock, cells recovered with 1 mL LB, incubated 1 h at 37° C., and then plated onto LB/Agar petri dishes carrying appropriate antibiotic

Step 2: Screening test involves picking 6 to 10 random colonies from agar plate and culturing them.

The cultures will be used to make glycerol stocks (for storage) and for enzyme assays

Step 3: For assays, colonies will be cultured at 30 C to 37° C. to an OD600 of 0.6 and expression of protein/enzyme induced by addition of IPTG.

Enzyme activity can be measured in 2 ways: A) substrates may be added to the culture medium or onto agar plates and colorimetry used to assess level of activity in comparison to a standard.

B) The cells will be lysed and enzyme/protein will be extracted and a reaction set up with appropriate substrates.

Activity will be assessed by HPLC or colorimetry against a standard.

Step 4: Colonies containing the plasmid with gene of interest which show maximum enzyme activity will be further processed.

The bacterial cells containing required mutations will be made into competent cells using standard protocols such as Calcium chloride

The competent cells from Round 1 will be used for the transformation of the next set of plasmids with mutations

Transformation will be done using standard protocol such as heat shock and recovery in LB for 1 hour at 37° C., and Steps 1 to 4 (i.e. transformation, colony picking, assay and competent cell preparation) will be repeated.

The choice of plasmids carrying the different mutations either site specific or random for each Round can be randomized too thereby increasing permutation and combination.

The DNA from colonies with maximum enzyme activity will be sequenced to get the number and location of mutations.

The sequence from the clones with good or high activity will be fed back into the AI of internally derived technology and algorithm to get the next round of experiments.

From the new set of insilico designs, sgRNA, Donor DNA will be made and cloned into appropriate pCas9 or dCas9 vectors or the engineered customized vectors for the next round of transformations.

### Transformation of Single Donor Plasmid Carrying all Mutations

This is the alternate method that will be tried. These experiments will be done

using a single donor plasmid carrying all the mutations either specific or random.

single base mutations that will be random will be cloned together into one plasmid with their respective sgRNAs and the dCas9 or engineered pdCas9 whilst all specific mutations will be cloned into one plasmid along with their respective sgRNAs, template donor DNA and Cas9 or engineered pCas9.

These will then be transformed sequentially into the receiving parental plasmid with gene of interest with assessment done in between.

### Assessment of Results

All processes will be assessed with assays done at each round. After each round, DNA analysis will be done using restriction enzyme digestion or Sanger sequencing techniques.

The efficiency and probability of mutations and the permutation combinations will also be calculated.

The genetic algorithm will be based on Permutation & combination of mutations generated. This in turn depends on number of experiments (as in number of tubes) and crossovers.

### Expected Outcomes

These experiments will create a library of colonies and enzyme variants which can be assessed.

FIG. 11 depicts the expected outcomes of the experiments.

The expected outcome of the transformation of various plasmids in different combinations can be calculated based on probability and efficiency of mutations being transferred from constructed plasmids (donors) to gene of interest in bacterial cells (receptor plasmids).

Several factors will affect the outcomes.

One is the concentration of plasmids: If similar concentration of plasmids are used, and assuming the lengths of target sgRNAs and PAMs are the same then the probability of all added mutations being transferred to the gene of interest would be equal.

If plasmids are transformed at different concentrations then the probability of plasmids with higher concentration transferring mutation may be higher than that of lesser concentration based on formula:
a. X=transformation threshold
b. Dam1, 2&3, 4>X
c. Plasmid intake Factor=a

There could also be an optimum concentration needed and therefore the mutations on plasmids at lower concentration might work better.

Outcome based on site of mutations: We will investigate whether the region of homology of target site whether—GC or AT rich will affect this probability.

This will also add to the permutation/combination of outcome.

Overall, even though the number of mutations incorporated is important and will be verified by Sanger sequencing, the main assessment will be the activity of protein or enzyme

### Outcome From Preliminary Experiments

Preliminary experiments were done to test outcome of transforming 2 plasmids into bacterial cells. We used the pET plasmid carrying the gene of interest and pUC18 plasmid in equal concentrations.

These were transformed into competent cells and plated onto agar plate containing kanamycin (for pET with gene of interest) and Ampicillin (test plasmid).

Colonies growing on double antibiotic plates were screened with restriction enzyme digestion.

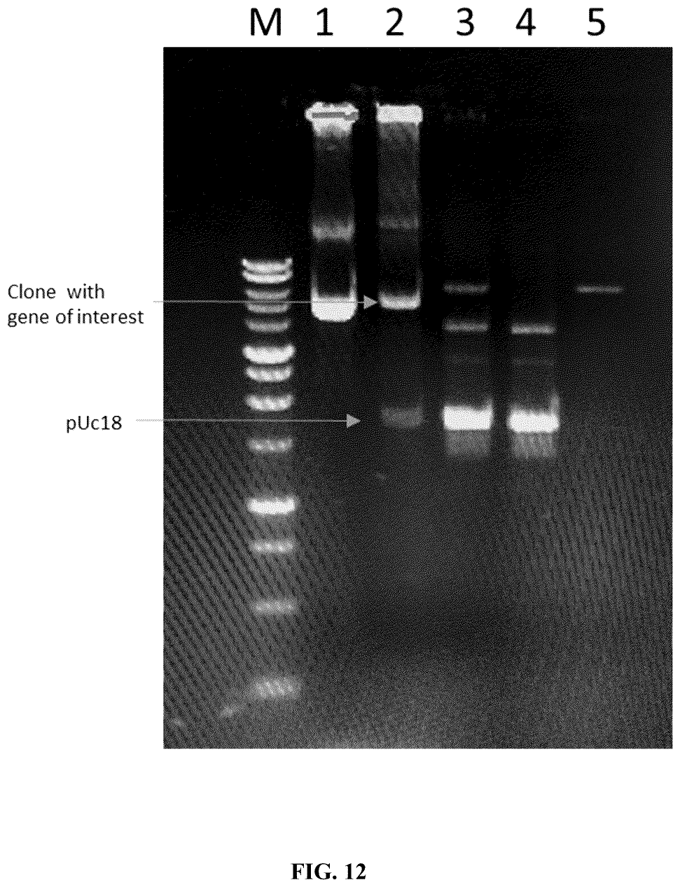

FIG. 12 shows an image of the agarose gel with these results.

We showed that 2 plasmids can be transformed (lane 2).

FIG. 12: Preliminary results of testing the transformation of 2 plasmids into bacteria.

Two plasmids; one pET carrying gene of interest (clone with gene of interest) and the other pUC18 were transformed into bacteria.

Plasmid DNA was isolated from colonies and restriction enzyme digested with XhoI.

Lane 1: shows DNA from colony transformed with pET with gene of interest only.

Lane 2: Plasmid DNAs from colony transformed with both plasmids (clone with gene of interest) and the other pUC18) showing characteristic bands of the two (blue arrows).

Lane 3: Mix of pure plasmid DNAs-pET with gene of interest and pUC18 digested with XhoI showing the two bands respectively.

Lane 4: pure pUC18 DNA digested with XhoI.

Lane: 5: pure plasmid clone (pET with gene of interest) digested with XhoI.

These results show that we could successfully transform cells with two different plasmids as in lane 2 we could see both plasmid bands.

Colorimetry assay developed in the lab has been used to test enzyme variants derived using the 7D grid technology (FIG. 9).

14 different variants were screened against different substrates and the range of colour from yellow to dark orange showed the level of activity.

We will use this developed assay to screen variants from CRISPR Cas experiments on agar plate as well as 96-well plates.

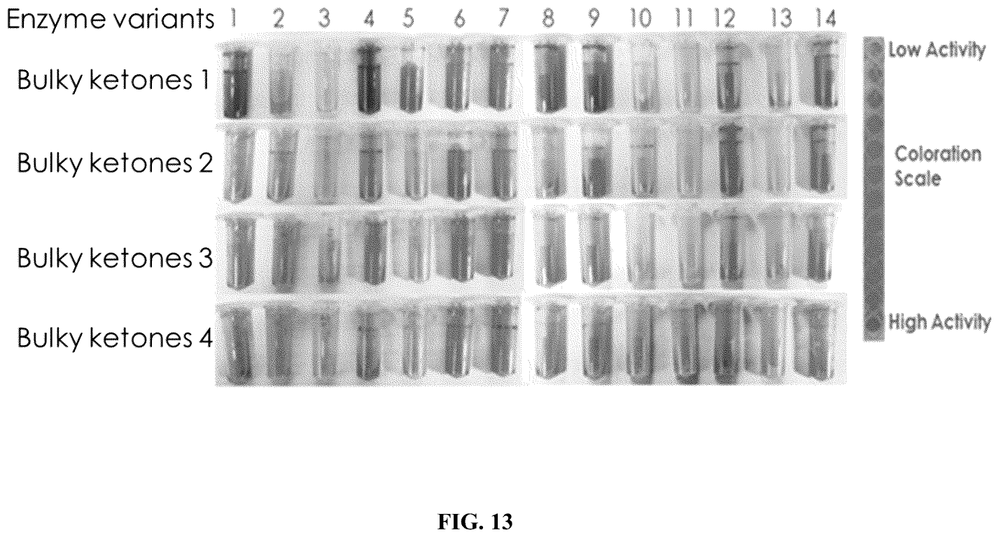

FIG. 13: Colorimetric based enzyme assay to check activity.

14 different representative transaminase variants were screened on the basis of their activity against four different ketones.

Red/orange coloration indicated transaminase activity against different substrates.

Intensity of orange to red colour showed the specificity and level of activity of the different transaminases towards different substrates; less coloration indicated moderate conversion.

This method will be used for verification of enzyme activity in tubes or on plates or on 96 well plates.

### SCOPE OF WORK WITH EXAMPLES

The scope of this work is far reaching and widely applicable.
d. Using insilico studies, hotspots will be identified for generating site specific and random mutations.
e. For example, a specific amino acid change at amino acid 18 and a random change anywhere between sites 40 and 65 has been identified to give higher efficiency of enzyme activity.
f. Many such hotspots will be identified for generating beneficial mutations which will help increase the activity of the protein and these will be constructed into CRISPR based vectors.
g. The genes of the enzymes are cloned into an expression plasmid and transformed into bacterial strain BL21.
h. To this bacterial strain which will be the receiving plasmid, 3-4 newly constructed plasmids carrying either specific or random mutations chosen from identified insilico studies, will be transformed.
i. Colonies picked will be analysed for enzyme activity using specific substrates in a reaction.
j. These can be further analyzed using colorimetry in either agar plate, media or tube/96 well plate or by HPLC.
k. The colonies or clones showing highest or best enzyme activity from the first round will then be made into competent cells and another round of transformation will be performed with a different set of donor plasmids carrying different combination of mutations.
l. Enzyme assays with Km and Kcat will be assessed and those clones/colonies showing better/best/highest activity will be analyzed by Sanger sequencing to verify the permutation and combination of mutations.
m. This incremental process can be used for any protein or enzyme to study and achieve highest enzyme activity or function.

### Applications

The above methodology can be and will be used for engineering proteins and enzymes in any or all of the applications mentioned above that are enzymes, antibodies, therapeutic proteins and enzymes and proteins important in any industrial and/or healthcare application.

These can include enzymes used to make Active Pharmaceutical Ingredient (API) such as ketoreductases, lipases, transaminases, Penicillin G acylases etc. as well as antibodies, various proteins and enzymes essential for important processes.

Abbreviations

| Abbre- | |
|---|---|
| viations | Definition |
| sgRNA | single guide Ribonucleic acid |
| PAM | Protospacer Adjacent Motif |
| D | random mutations performed by single |
| | base substitutions using Deaminase |
| AA | Amino Acid |
| p | Plasmid |
| API | Active Pharmaceutical Ingredient |
| LB | Luria broth |
| IPTG | Isopropyl ß-D-1-thiogalactopyranoside |
| MD | Molecular Dynamics |
| QM | Quantum Mechanics |
| MM | Molecular Mechanics |
| QM/MM | Quantum Mechanics hybridized with Molecular Mechanics |
| SSM | Site Saturated Mutagenesis |
| PHP | Probe based Hotspot Predictions |
| NMA | Normal Mode Analysis |
| ED | Ensemble Docking |
| Name | Definition |
| 1. Mole- | Molecular Dynamics simulations is a Computer Simulation |
| cular | method for analyzing the physical movement of atoms and |
| Dynamics | molecules in three-dimensional space. One of the principal |
| (MD) | methods in the theoretical study |
| | of biological molecules. Here the simulation of |
| | protein motion is realized by the numerical |
| | solution of the classical Newtonian dynamic equations. |
| | MD simulations provide detailed information |
| | on the fluctuations and conformational changes |
| | of proteins and nucleic acids. These |
| | conformational changes of proteins |
| | and nucleic acids is believed |
| | to mimic their behavior in the natural system. |
| | These methods are now routinely used to |
| | investigate the structure, dynamics and |
| | thermodynamics of biological molecules |
| | and their complexes |
| 2. | Quantum Mechanics hybridized with Molecular |
| Quantum | (mechanics QM/MM) simulations is used to |
| Mechanics | investigate a chemical reaction |
| (QM/MM) | or process at the appropriate level of quantum |
| | chemistry theory. In QM/MM methods, |
| | the region of the system in which the |
| | enzymatic reaction takes place is treated at |
| | an appropriate level of quantum chemistry |
| | theory (QM region), while the remainder is described |
| | by a molecular mechanics force field(MM region). |
| | This method combines the strength |
| | of accuracy (ab initio QM |
| | calculations) and speed ( Molecular mechanics). The |
| | hybrid QM/MM calculations gives us energy calculations |
| | of three classes of interactions: interactions |
| | between atoms in the QM |
| | region, between atoms in the MM region and interactions |
| | between QM and MM atoms. |
| | Within this approach, chemical |
| | reactivity can be studied in large systems of enzymes and |
| | proteins |
| 3. CAVER | CAVER (Damborsky et. al. 2018) is a |
| | software tool for analysis and visualization |
| | of tunnels, channels and cavities in protein |
| | structures. Here identified protein pockets, tunnels are |
| | characterized by residues lining them which |
| | are important for drug design and |
| | molecular enzymology. These identifications |
| | of tunnels, pockets and channels are calculated |
| | in static and dynamic structures |
| 4. Trj | Trj_cavity (Bond et. al., 2014) is a tool |
| Cavity | that is used to characterize and identify cavities |
| | from trajectories or stand-alone PDB files. |
| | The tool provides output as a static pdb and a trajectory to |
| | visualize the change in cavities across time. |
| | The dynamic nature of cavities could be used |
| | to understand the nature of the residues |
| | lining the pockets and provide insight into engineering |
| | of the enzyme for improved stability or activity |
| 5. SSM | Site Saturated Mutagenesis (SSM) is an experimental |
| | design developed in house by Kcat to screen through |
| | each residue that is considered as a hotspot for |
| | engineering in silico to yield better enzyme activity. |
| | The selected residue/hotspot is changed to all |
| | other possible residues that can occur in the protein as |
| | point mutation by the use of computational methods |
| | and then taken to Molecular dynamics |
| | simulations to equilibrate and obtain the |
| | dynamic changes brought about by the |
| | mutation. The results of the simulation is then |
| | used to understand the effectiveness of the |
| | mutation(s) on the enzyme activity |
| 6. PHP | Probe-based Hotspot Prediction is a method by which |
| | enzymes can be screened for activity based on the |
| | nature of change that is required in the mutation |
| | site. The method, developed in-house |
| | by Kcat, involves erasing the side-chain atoms of residue |
| | hotspots and placing probes that mimic the physical and |
| | chemical nature of amino acids to achieve the desired |
| | change in the active site. The probes and the protein |
| | are then subjected to differing conditions of |
| | temperature to induce a “heat shock” that will give |
| | unique conformations of the residues around the probe |
| | site which provides an understanding of the nature of |
| | interactions of the residues and the mutation and |
| | such the entire enzyme |
| 7. Kcat | Contact score algorithm is used to measure, score and |
| Contact | rank the physical contacts or interactions that occur |
| Score | between a target residue/ligand and its surrounding |
| algorithm | residues. The algorithm takes |
| | the number of interactions & the distance between the |
| | interacting residues and scores it |
| | as a means of quantifying the |
| | interaction. The score can then be used to rank the |
| | interactions a particular residue makes with the target |
| 8. 7D- | A proprietory method for protein engineering that |
| Grid | constructs a three-dimensional gridspace around a |
| Tech- | protein. Mutations are introduced based of calculation |
| nology | of pair interaction energies by FMO method |

What is claimed is:

1. A process for introducing multiple mutations into a gene of interest located within Plasmid DNA that is transformed into a bacterial cell using engineered CRISPR-Cas technology, the process comprising:
a) identifying hotspots in the gene of interest in the Plasmid DNA (expression plasmid) of the bacterial cell;
b) incorporating multiple random mutations in a vector comprising dCas9 (Deaminase single base mutations) or a customized vector, wherein the customized vector used for random mutations comprises an engineered dcas9 enzymes and one or more sgRNA(s);
c) transforming the vector obtained in step b in the bacterial cell comprising the gene of interest in the Plasmid DNA (expression plasmid); and
d) introducing pCas9 vector or customized vector (designed for the specific mutation) with multiple specific mutations into competent bacterial cell such that the gene of interest in the Plasmid DNA of the bacterial cell comprises both the specific mutations and random mutations, wherein the customized vector for specific mutations comprises an engineered Cas9 enzyme and one or more sgRNA(s) in combination with one or more donor DNA(s).
e) identifying hotspots in the gene of interest where the hotspots include specific mutations and designated regions for random mutations in the enzyme encoded by the gene of interest wherein, the respective mutations are encoded in donor DNA and guided by the sgRNA that are inserted in engineered pCas9 plasmids;
characterized in that the process comprises:
f) constructing a first engineered pCas9 plasmid with one or more sgRNAs that corresponds to the regions for random mutagenesis derived from step (a), and the engineered pCas9 plasmid contains a J23119 promoter upstream of the sgRNAs and a lac promoter upstream of the gene encoding engineered dCas9 enzymes, and a temperature-inducible lambda operator upstream of both components, wherein the gene encoding the engineered dCas9 enzymes are mutated with reference to the naturally occurring dCas9 and is fused with a gene encoding deaminase enzyme containing mutations with reference to the naturally occurring gene encoding deaminase enzyme;
g) transforming a bacterial cell with the first engineered pCas9 plasmid obtained in step b resulting in the bacterial cell being transfected with both, the plasmid DNA carrying the gene of interest and the first engineered pCas9 plasmid; and
h) transforming a second engineered pCas9 plasmid into the bacterial cell obtained in step c, to induce multiple specific mutations in the gene of interest in the plasmid DNA, wherein the second engineered pCas9 plasmid contains a gene encoding engineered Cas9 enzyme, one or more sgRNAs, and one or more donor DNAs that corresponds to the specific mutations obtained in step (a), and is characterized by a leader sequence upstream to the sgRNAs and donor DNAs and a tet promoter upstream of the gene encoding engineered Cas9 enzyme, wherein the gene encoding engineered Cas9 enzyme has deletions and substitution mutations with reference to the naturally occurring gene that encodes Cas9 enzyme.

2. The process as claimed in claim 1, wherein the plasmid DNA, which harbors the gene of interest, along with the engineered plasmids that contain both engineered Cas9 and engineered dCas9 enzymes, are concurrently introduced into the bacterial cell by means of transformation techniques to ensure the simultaneous presence of all three plasmids within the bacterial cell.

3. The process as claimed in claim 1, wherein the bacterial cell with the three plasmids functioning as a system wherein engineered dCas9 enzymes, assisted by respective sgRNAs, bind to the gene of interest for single base editing using fused deaminase, and the engineered Cas9 enzymes, guided by respective sgRNAs, bind to the gene of interest, thereby incorporating random and specific mutations within distinct areas of the gene of interest.
